(19) United States
(12) Patent Application Publication
(10) Pub. No.: US 2016/0325048 A1
(43) Pub. Date: Nov. 10, 2016

(54) UNIVERSAL PLUNGER RODS, SYSTEMS, AND METHODS OF USE AND ASSEMBLY

(71) Applicant: Regeneron Pharmaceuticals, Inc., Tarrytown, NY (US)

(72) Inventors: Joseph BERRY, Neshanic Station, NJ (US); Michael CUPICHA, East Schodack, NY (US); Alexei GORALTCHOUK, Cambridge, MA (US); Mykhaylo HRYTSAK, Hastings-on-Hudson, NY (US); Courtney NICHOLAS, East Greenbush, NY (US)

(73) Assignee: Regeneron Pharmaceuticals, Inc., Tarrytown, NY (US)

(21) Appl. No.: 15/145,922

(22) Filed: May 4, 2016

Related U.S. Application Data
(60) Provisional application No. 62/158,346, filed on May 7, 2015.

(51) Int. Cl. A61M 5/315

(52) U.S. Cl. CPC A61M 5/31513; A61M 5/31515; A61M 2005/31521; A61M 2207/00

(57) ABSTRACT

The present disclosure provides plunger rods including a shaft, an injection member and a contact member. The shaft has a first end and a second end, and the injection member is coupled to the shaft at the first end. The contact member is coupled to the shaft at the second end. The present disclosure also provides injection systems including a container, a piston and a plunger. The container has with a first end and a second end. The piston is positioned within the container, and the plunger rod is positioned within the container adjacent to the piston. The present disclosure also provides methods of assembling an injection system. The methods include obtaining a container, a piston, and a plunger rod. The methods further include inserting the piston into an opening in a second end of the container, and inserting the plunger rod into the opening in the second end of the container and positioning the plunger rod adjacent to the piston.

### CROSS REFERENCE TO RELATED APPLICATION

This application claims priority benefit under 35 U.S.C. §119(e) to U.S. Provisional Application No. 62/158,346, filed May 7, 2015, and entitled Universal Plunger Rods, Systems, And Methods of Use and Assembly, the entirety of which is incorporated herein by reference in its entirety.

### FIELD OF THE INVENTION

The present invention relates generally to drug delivery systems for administering medication. More specifically, but not exclusively, the present invention concerns plunger rods, plunger rod systems, and injection systems.

### BACKGROUND OF THE INVENTION

Many injectors and assisted injection systems use a piston head and plunger rod system to deliver medication to a patient. The currently available piston head and plunger rod systems may experience bacteria build up along the plunger head. The bacteria build up may cause contamination of the medication or fluid being administered to the patient when force is applied to the plunger rod. The bacteria build up may also cause coupling problems between the piston head and the end of the plunger rod. In addition, the currently available piston head and plunger rod systems are generally either a screw type plunger rod which screws into a threaded opening in the piston head or a recessed type where the plunger rod is held in place by a smooth recessed area in the piston head. If the wrong size plunger rod is used for either the screw type or recessed type plunger rods, the resulting motion of the piston head may be erratic due to plunger rod and piston head misalignment. The erratic motion may cause plunger rod slippage and movement that may result in variable dose administration pressure to the piston. Thus, piston head and plunger rod systems that prevent contamination of the medication being administered and provide for uniform administration pressure are needed.

### SUMMARY OF THE INVENTION

Aspects of the present invention provide plunger rods, plunger rod systems, and injection systems. The present invention also provides methods for assembling and using the plunger rods, plunger rod systems, and injection systems.

In one aspect provided herein is a plunger rod including a shaft with a first end and a second end, an injection member coupled to the shaft at the first end, and a contact member coupled to the shaft at the second end.

In another aspect, provided herein is plunger rod system including a piston and a plunger rod positioned adjacent to the piston.

In yet another aspect, an injection system including a container with a first end and a second end, a piston positioned within the container, and a plunger rod positioned within the container adjacent to the piston.

In a further aspect, provided herein is a method of assembling an injection system, the method includes obtaining a container, a piston, and a plunger rod. The method also includes inserting the piston into an opening in a second end of the container. The method further includes inserting the plunger rod into the opening in the second end of the container and positioning the plunger rod adjacent to the piston.

These, and other objects, features and advantages of this invention will become apparent from the following detailed description of the various aspects of the invention taken in conjunction with the accompanying drawings.

### BRIEF DESCRIPTION OF DRAWINGS

The accompanying drawings, which are incorporated in and constitute a part of the specification, illustrate embodiments of the invention and together with the detailed description herein, serve to explain the principles of the invention. The drawings are only for purposes of illustrating preferred embodiments and are not to be construed as limiting the invention. It is emphasized that, in accordance with the standard practice in the industry, various features are not drawn to scale. In fact, the dimensions of the various features may be arbitrarily increased or reduced for clarity of discussion. The foregoing and other objects, features and advantages of the invention are apparent from the following detailed description taken in conjunction with the accompanying drawings in which:

FIG. 1 is a perspective view of a plunger rod system, in accordance with an aspect of the present invention;

FIG. 2 is a side view of the plunger rod system of FIG. 1, in accordance with an aspect of the present invention;

FIG. 3 is an exploded perspective view of the plunger rod system of FIG. 1, in accordance with an aspect of the present invention;

FIG. 4 is a perspective view of the piston of the plunger rod system of FIG. 1, in accordance with an aspect of the present invention;

FIG. 5 is a side view of the piston of FIG. 4, in accordance with an aspect of the present invention;

FIG. 6 is a front perspective view of the piston of FIG. 4, in accordance with an aspect of the present invention;

FIG. 7 is a back perspective view of the piston of FIG. 4, in accordance with an aspect of the present invention;

FIG. 8 is a side view of the plunger rod of the plunger rod system of FIG. 1, in accordance with an aspect of the present invention;

FIG. 9 is a perspective view of alternative plunger rods, in accordance with an aspect of the present invention;

FIG. 10 is a perspective view of another plunger rod system, in accordance with an aspect of the present invention;

FIG. 11 is a side view of the plunger rod system of FIG. 10, in accordance with an aspect of the present invention;

FIG. 12 is an exploded, perspective view of the plunger rod system of FIG. 10, in accordance with an aspect of the present invention;

FIG. 13 is a side view of an injection system including the plunger rod system of FIG. 1 in a first injection position, in accordance with an aspect of the present invention;

FIG. 14 is a perspective view the injection system of FIG. 13, in accordance with an aspect of the present invention; and

FIG. 15 is a side view of the injection system of FIG. 13 in a second injection position, in accordance with an aspect of the present invention.

### DETAILED DESCRIPTION FOR CARRYING OUT THE INVENTION

Generally stated, disclosed herein are plunger rods, plunger rod systems, and injection systems. Further, methods of assembling and using the plunger rods, plunger rod systems, and injection systems are discussed.

In this detailed description and the following claims, the words proximal, distal, anterior, posterior, medial, lateral, superior and inferior are defined by their standard usage for indicating a particular part of a device according to the relative disposition of the device with respect to a body or directional terms of reference. For example, “proximal” means the portion of a device nearest the point of injection, while “distal” indicates the portion of the device farthest from the point of injection. As for directional terms, “anterior” is a direction towards the front side of the device, “posterior” means a direction towards the back side of the device, “medial” means towards the midline of the device, “lateral” is a direction towards the sides or away from the midline of the device, “superior” means a direction above and “inferior” means a direction below another object or structure.

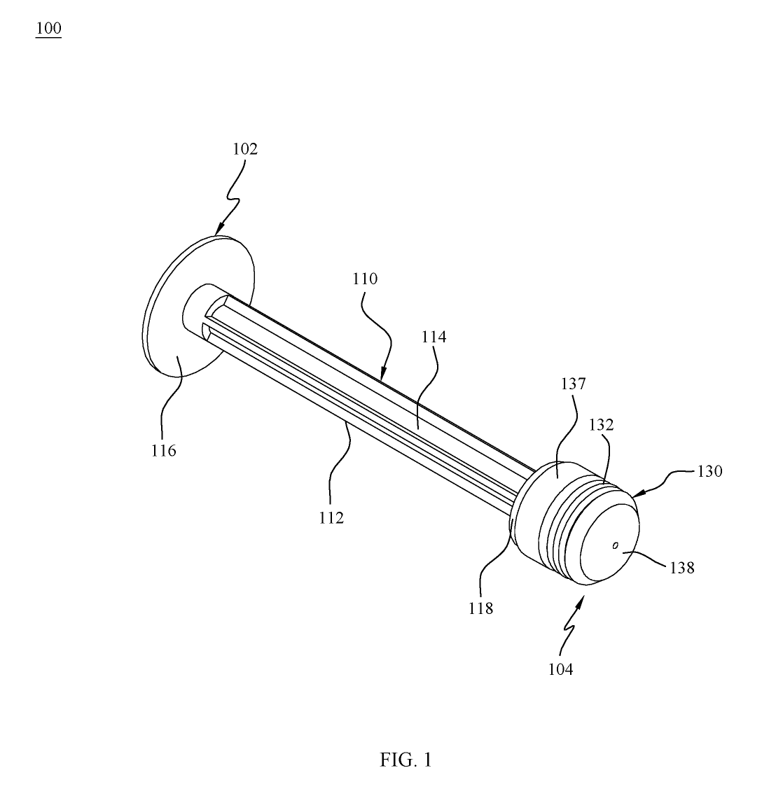

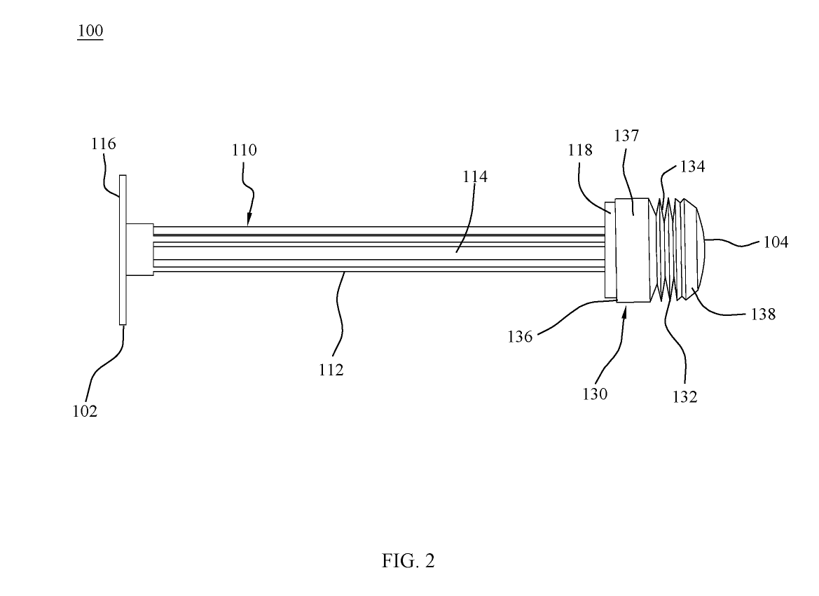

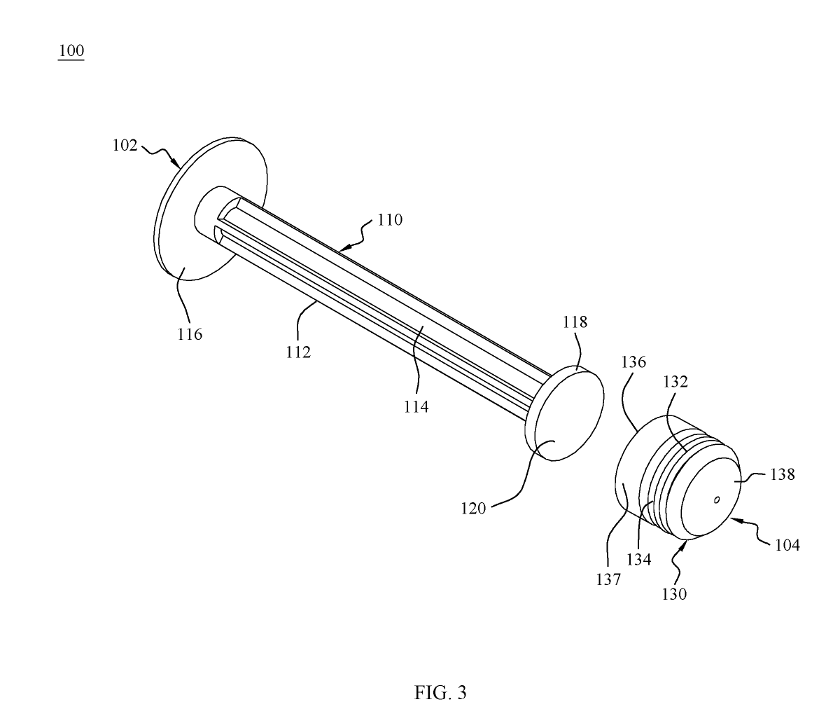

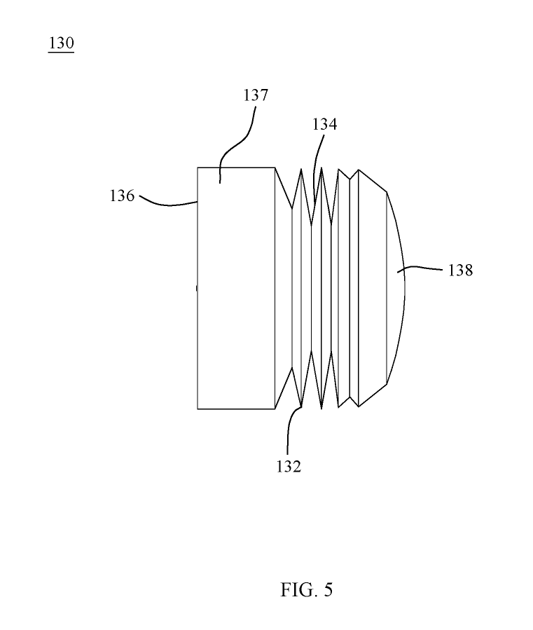

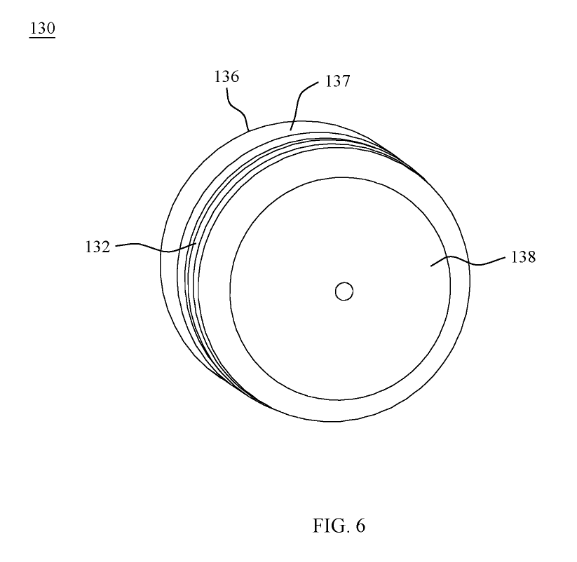

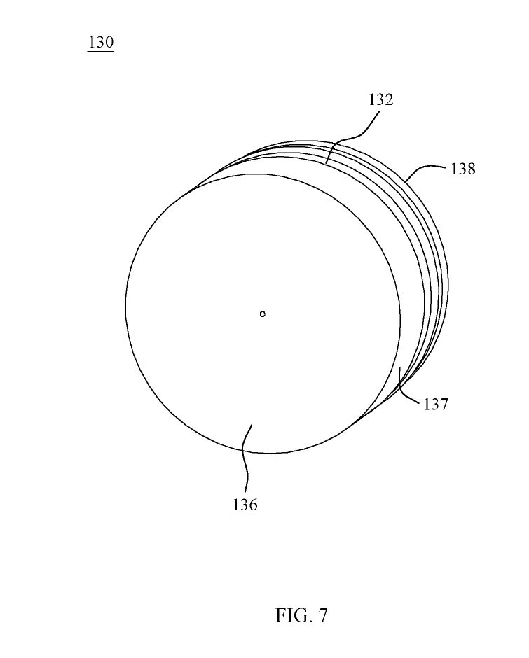

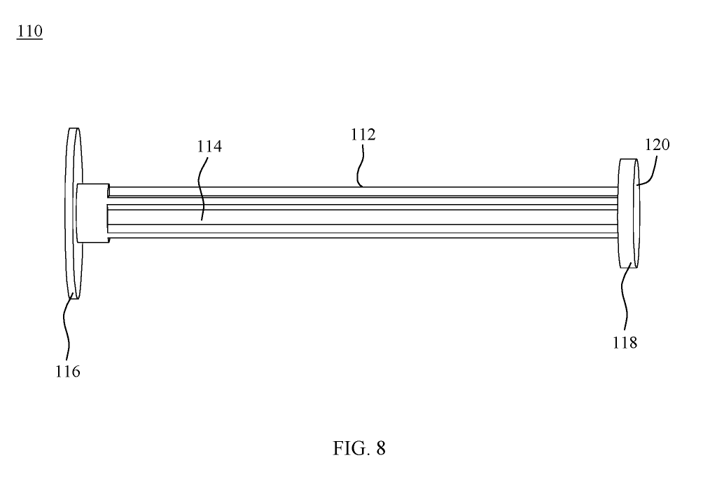

Referring to the drawings, wherein like reference numerals are used to indicate like or analogous components throughout the several views, and with particular reference to FIGS. 1-8, there is illustrated a plunger rod system 100. The plunger rod system 100 may include a plunger rod 110 and a piston 130. The plunger rod 110 may include a shaft 112, an injection member 116, and a piston contact member 118. The shaft 112 may have, for example, at least one groove 114 inset into the shaft 112 and extending along the length of the shaft 112. In the depicted embodiment, the shaft 112 includes three grooves 114. The at least one groove 114 may extend from the piston contact member 118 along the length of the shaft 112 and terminate before reaching the injection member 116. The injection member 116 may be positioned at a first end 102 of the plunger rod system 100 and the piston 130 positioned at the second end 104 of the plunger rod system 100. The piston contact member 118 may include a contact surface 120 on a side of the contact member 118 opposite the shaft 112, as shown in FIG. 8. The piston contact member 118 may be sized and shaped to completely contact an end 136 of the piston 130. The piston contact member 118 may, for example, include at least a portion fabricated from an elastomeric or deformable material. Preferably, the outer rim of the piston contact member 118 is made of the elastomeric or deformable material to engage the interior surface of the syringe for stability and sealing purposes during injection. The contact member 118 may be, for example, sufficiently stiff to enable movement of the piston 130 and allow for air between the contact surface 120 and the end 136 to escape as the contact member 118 translates the piston 130. The air may escape between the outer diameter of the contact member 118 and the inner diameter of the barrel or through holes (not shown) within the contact member 118. The plunger rod 110 for a 1 mL long syringe may have, for example, an injection member 116 with a diameter ranging from approximately 7 mm to 25 mm, a shaft 112 with an outer diameter of approximately 1 mm to 6 mm, and a contact member 118 with a diameter slightly larger than the inner diameter of the syringe or cartridge barrel into which the plunger rod 110 is inserted.

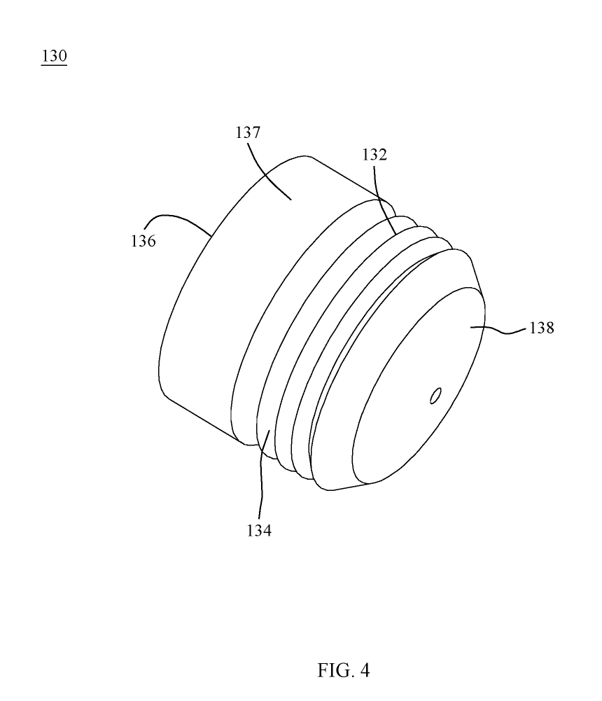

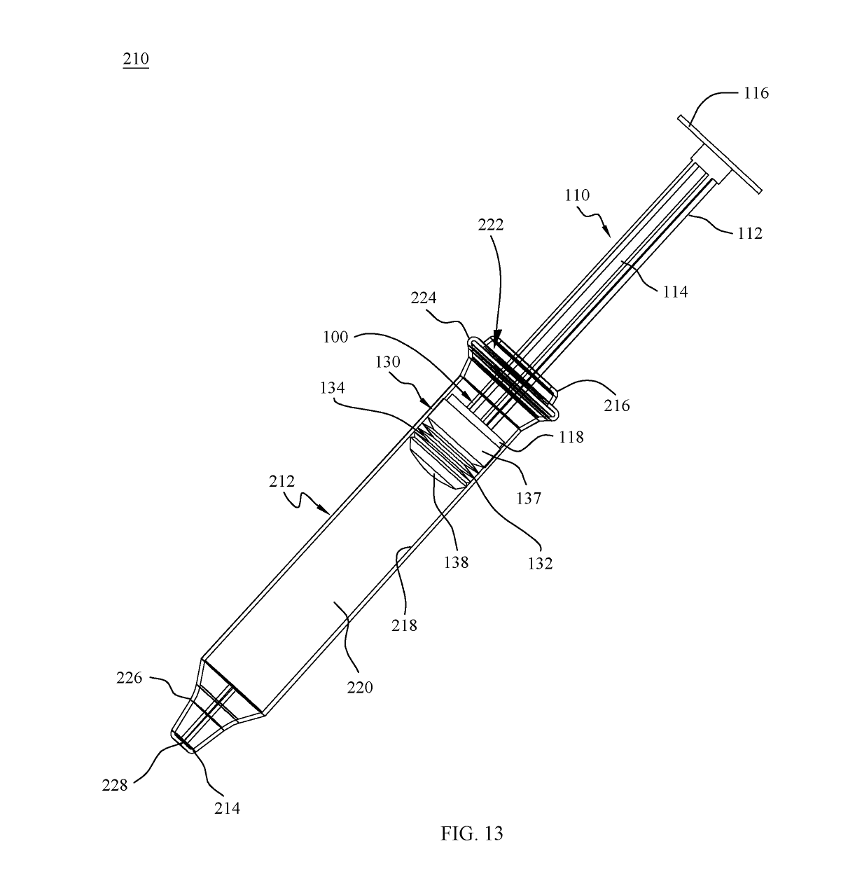

As shown in FIGS. 4-7, the piston 130 may include, for example, a first base portion 137 with a contact end 136 and a second base portion 138 at the second end 104 of the plunger rod system 100. Although not shown, the contact end 136 may include, for example, an opening extending into the piston 130. The first base portion 137 may be, for example, thicker than the second base portion 138. The first base portion 137 may be, for example, elongated to eliminate backflow contamination during administration of a medication or fluid to a patient. The piston 130 may also include at least one flange member 132 and at least one recess or groove 134 positioned between the first base portion 137 at the first end of the piston 130 and the second base portion 138 at the second end of the piston 130. The first base portion 137 and second base portion 138 may be sized to be received within a syringe, container, cartridge, or vial, as shown in FIGS. 13-15. The terms “syringe,” “container,” “cartridge,” and “vial” may be used interchangeably herein as they each essentially refer to a means for holding and administering a medication or fluid to a patient. The first base portion 137 and second base portion 138 may be sized to prevent medication from passing to the distal end of the container during injection through a fluid pathway at the proximal end of the container. The piston 130 may prevent medication from passing to the distal end of the container to maintain the sterility of the medication. It is also contemplated that the piston 130 may include a plurality of flange members 132. The number of flange members 132 may range from, for example, the minimum number of flange members 132 necessary to affect a seal and prevent fluid from passing through the piston 130 to the maximum number of flange members 132 which still allow the piston 130 to slide within the container. In one embodiment, the piston 130 may include, for example, approximately two to five flange members 132. The outer diameter of the piston 130 may be, for example, slightly larger than the inner diameter of the syringe or cartridge barrel in which the piston 130 is being inserted to form a seal between the piston 130 and the interior surface of the barrel.

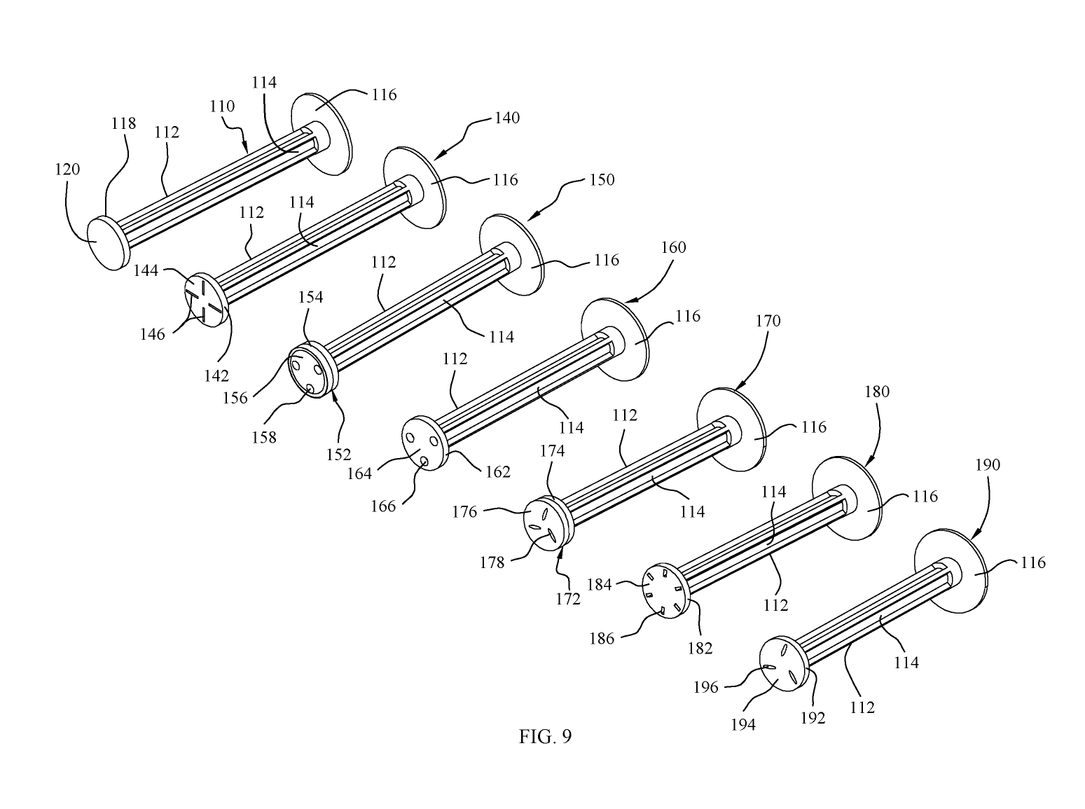

Referring now to FIG. 9, several plunger rod embodiments are shown. The plunger rod 140 may include, for example, a shaft 112, an injection member 116, and a contact member 142. The shaft 112 and injection member 116 are of the type described in greater detail above with reference to the plunger rod 110. The contact member 142 may include a contact surface 144 positioned on the side of the contact member 142 opposite the shaft 112. The contact surface 144 may be sized to engage the contact surface 136 of the piston 130. The contact member 142 may also include at least one opening or slot 146. As illustrated, the at least one opening or slot 146 may be, for example, four openings or slots 146.

The plunger rod 150 may include, for example, a shaft 112, an injection member 116, and a contact member 152. The shaft 112 and injection member 116 are of the type described in greater detail above with reference to the plunger rod 110. The contact member 152 may include a support ring 154 coupled to the shaft 112 and a contact surface 156 positioned on the side of the support ring 154 opposite the shaft 112. The support ring 154 may be a separate or integral part of the contact member 152. The support ring 154 may also be made of the same or a different material than the contact surface 156 portion of the plunger rod 150 to provide the plunger rod 150 with additional support, stability, and sealing. Although only one support ring 154 is shown, additional support rings along the length of the shaft 112 are also contemplated, for example, to provide stability and prevent bending or flexion along the shaft 112. The contact surface 156 may be sized to engage the contact surface 136 of the piston 130. The contact member 152 may also include a rim or flange around the circumference of the contact surface 156 and at least one opening 158. The rim or flange may be, for example, a rubber or other elastomeric or deformable material and the contact surface 156 may be, for example, a rigid material. A contact member 152 with a rim or flange may be used, for example, to engage the interior surface of the syringe barrel to provide additional stability and form an additional seal, while allowing air to escape between the surface of the contact member 152 and the end 136 of the piston 130. As illustrated, the at least one opening 158 may be, for example, three openings 158.

The plunger rod 160 may include, for example, a shaft 112, an injection member 116, and a contact member 162. The shaft 112 and injection member 116 are of the type described in greater detail above with reference to the plunger rod 110. The contact member 162 may include a contact surface 164 positioned on the side of the contact member 162 opposite the shaft 112. The contact surface 164 may be sized to engage the contact surface 136 of the piston 130. The contact member 162 may also include at least one opening 166. As illustrated, the at least one opening 166 may be, for example, three openings 166.

The plunger rod 170 may include, for example, a shaft 112, an injection member 116, and a contact member 172. The shaft 112 and injection member 116 are of the type described in greater detail above with reference to the plunger rod 110. The contact member 172 may include a support ring 174 coupled to the shaft 112 and a contact surface 176 positioned on the side of the support ring 174 opposite the shaft 112. The support ring 174 may be a separate or integral part of the contact member 172. The support ring 174 may also be made of the same or a different material than the contact surface 176 portion of the plunger rod 170 to provide the plunger rod 170 with additional stability, support, and sealing. The contact surface 176 may be sized to engage the contact surface 136 of the piston 130. The contact member 172 may also include at least one opening or slot 178. As illustrated, the at least one opening or slot 178 may be, for example, three openings 178.

The plunger rod 180 may include, for example, a shaft 112, an injection member 116, and a contact member 182. The shaft 112 and injection member 116 are of the type described in greater detail above with reference to the plunger rod 110. The contact member 182 may include a contact surface 184 positioned on the side of the contact member 182 opposite the shaft 112. The contact surface 184 may be sized to engage the contact surface 136 of the piston 130. The contact member 182 may also include at least one opening or slot 186. As illustrated, the at least one opening or slot 186 may be, for example, six openings 186.

The plunger rod 190 may include, for example, a shaft 112, an injection member 116, and a contact member 192. The shaft 112 and injection member 116 are of the type described in greater detail above with reference to the plunger rod 110. The contact member 192 may include a contact surface 194 positioned on the side of the contact member 192 opposite the shaft 112. The contact surface 194 may be sized to engage the contact surface 136 of the piston 130. The contact member 192 may also include at least one opening or slot 196. As illustrated, the at least one opening or slot 196 may be, for example, three openings 196.

The openings or relief ports 146, 158, 166, 178, 186, and 196 allow for air trapped between the plunger rod 110 and the piston 130 to exhaust through the contact member 118 of the plunger rod 110. The openings 146, 158, 166, 178, 186, and 196 may be positioned in various patterns and have various shapes and configurations to allow trapped air to escape and the plunger rod 110 to provide smooth and steady force on the piston 130. If the piston 130 includes an opening in the end 136, then the openings 146, 158, 166, 178, 186, and 196 may, for example, overlap with at least a portion of the piston opening to allow for air to escape from the piston opening as the piston 130 is translated.

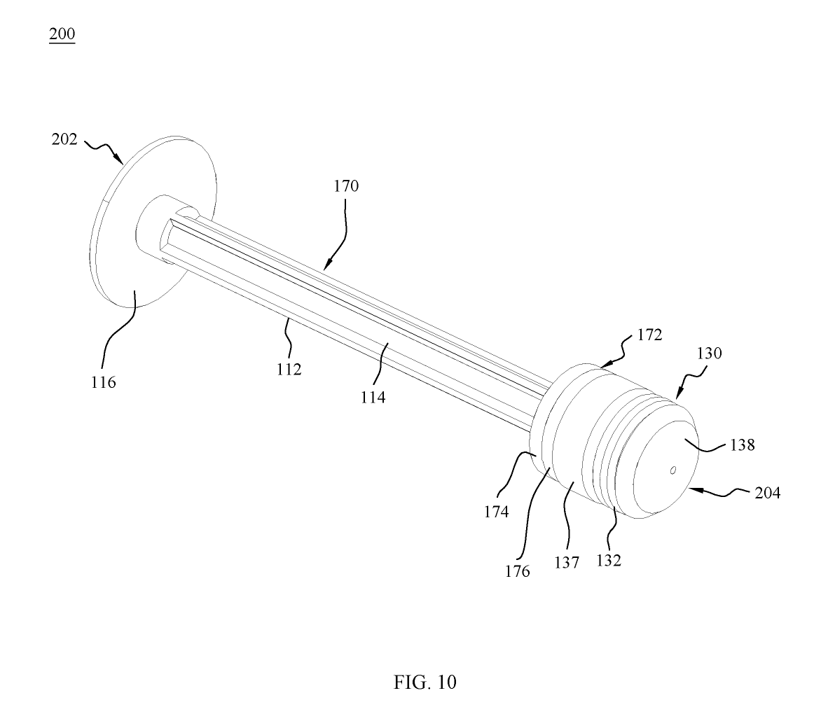

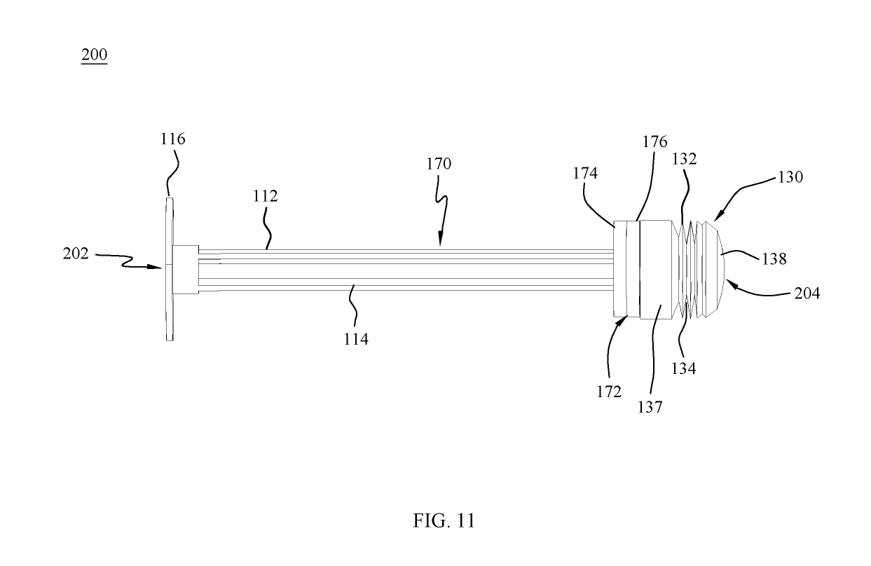

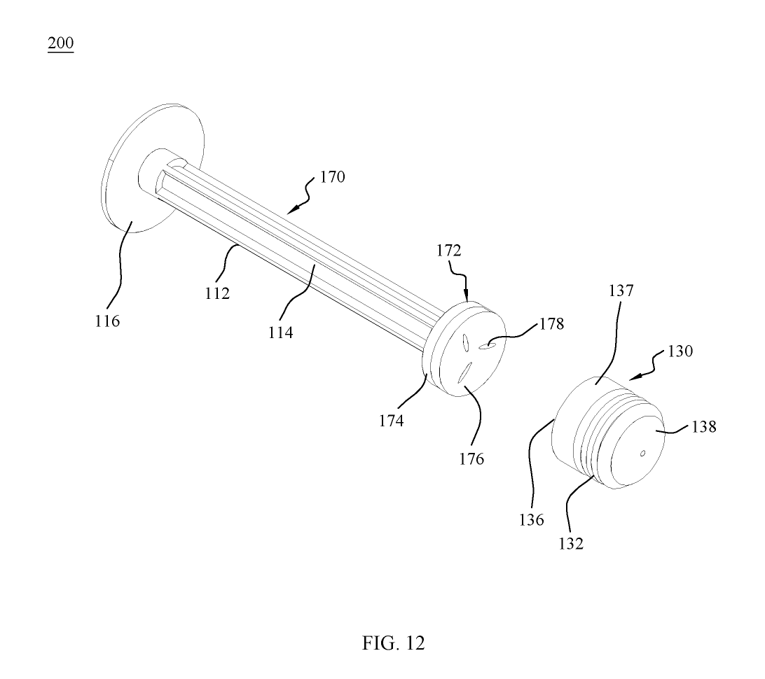

FIGS. 10-12 show another plunger rod system 200. The plunger rod system 200 may include the plunger rod 170 and the piston 130. The plunger rod 170 and piston 130 may be of the types described above with reference to FIGS. 4-7 and 9. The contact surface 176 engages the contact surface 136 during an injection to move the piston 130 as force is applied to the plunger rod 170. In addition, as force is applied to the plunger rod 170 during an injection, any air trapped between the plunger rod 170 and the piston 130 may escape through the openings 178 which extend through the contact end 172 of the plunger rod 170.

Referring now to FIGS. 13-15, an injection system 210 is shown. The injection system 210 may include a plunger rod 110, 140, 150, 160, 170, 180, 190, a piston 130, and a container 212. The plunger rods 110, 140, 150, 160, 170, 180, 190 and piston 130 may be of the type described above, which will not be described again here for brevity sake. The container 212 may be, for example, a cartridge, syringe, chamber, vial, or the like for receiving a medication or fluid for injection into a patient. The container 212 may include a first end 214 and a second end 216. The container 212 may also include a sidewall 218 forming a chamber 220. The container 212 may further include an opening 222 at the second end 216 that extends into the chamber 220. In addition, the second end 216 may include, for example, a flange 224 around the exterior circumference of the container 212. The first end 214 may include an injection tip 226 with an injection mechanism opening 228 to allow for administration of a medication or fluid to a patient. The injection mechanism opening 228 may extend from an exterior surface of the container 212 into the chamber 220 of the container 212. The injection mechanism opening 228 may receive, for example, a needle, tube, cannula, or the like to transfer the medication or fluid from the chamber 220 of the container 212 to a patient.

The injection system 210 may be assembled by obtaining a plunger rod 110, 140, 150, 160, 170, 180, 190, a piston 130, and a container 212. The piston 130 may be inserted into the container 212 by aligning the second base portion 138 with the opening 222 of the container 212 and sliding the piston 130 into the chamber 220. The first base portion 137, second base portion 138, and at least one flange member 132 of the piston 130 may be sized to engage the sidewall 218 of the container 212 and assist with preventing the medication from passing by the piston head member 130 toward the distal end of the container 212 to ensure the proper dosage of medication is delivered to the patient. The circumference and space between the at least one flange member 132 and first and second base portions 137, 138 allows for maximum contact between the at least one flange member 132 and the sidewall 218 of the container 212. Once the piston 130 is positioned within the chamber 220 of the container 212, the contact member 118 of the plunger rod 110 may be inserted into the opening 222 in the container 212 and translated until contact is made with the contact surface 136 of the piston 130. The contact member 118 may be sized to freely translate within the chamber 220 while also contacting the sidewalls 218 of the container 212 to allow for a smooth and steady injection. The chamber 220 may be filled with the desired amount of medication before or after insertion of the piston 130 and plunger rod 110 by known filling methods.

The injection system 210 may be used to administer medication to a patient by first coupling an injection mechanism (not shown) to the injection mechanism opening 228 of the container 212 at a first end. The second end of the injection mechanism may be coupled to a patient or a device already coupled to the patient. Next, the plunger rod 110 may be positioned in the container 212 and force may be applied to the injection member 116 of the plunger rod 110. As force is applied to the injection member 116, the force may be transferred to the piston contact member 118 which in turn applies force to the piston 130. The piston contact member 118 may be shaped to provide for the surface of contact member 118 to remain uniform as the piston 130 moves through the container 212 preventing kinking or excessive glide force. In addition, the piston contact member 118 may be shaped to allow for any air trapped between the piston 130 and plunger rod 110 to escape.

In one embodiment, for example, the shape of the piston contact member 118 may match the shape and approximate size of the contact surface 136 of the piston 130 to allow for a uniform force to be applied directly to the piston 130. Alternative shapes of the contact member 118 are also contemplated including, for example, a dome shape, an inverse dome shape, and a central protrusion (not shown) on the contact surface 120. The central protrusion may be positioned to engage an optional opening in the piston 130. The central protrusion of the contact member 118 may be sized, for example, to be the same size, smaller, or larger than the diameter of a central opening (not shown) extending into the piston 130 from the contact surface 136 when the piston contact member 118 engages the piston 130. The central protrusion of the contact member 118 may also have a length, for example, that is the same, longer, or shorter than the depth of the central opening (not shown) in the piston 130. The central protrusion of the contact member 118 may have, for example, a flat or cone shaped engagement end. When the central protrusion of the contact member 118 is longer than the depth of the central opening in the piston 130 or the diameter of the contact member 118 is larger than the diameter of the central opening of the piston 130, the contact surface 120 will float or suspend above the end 136 of the piston 130.

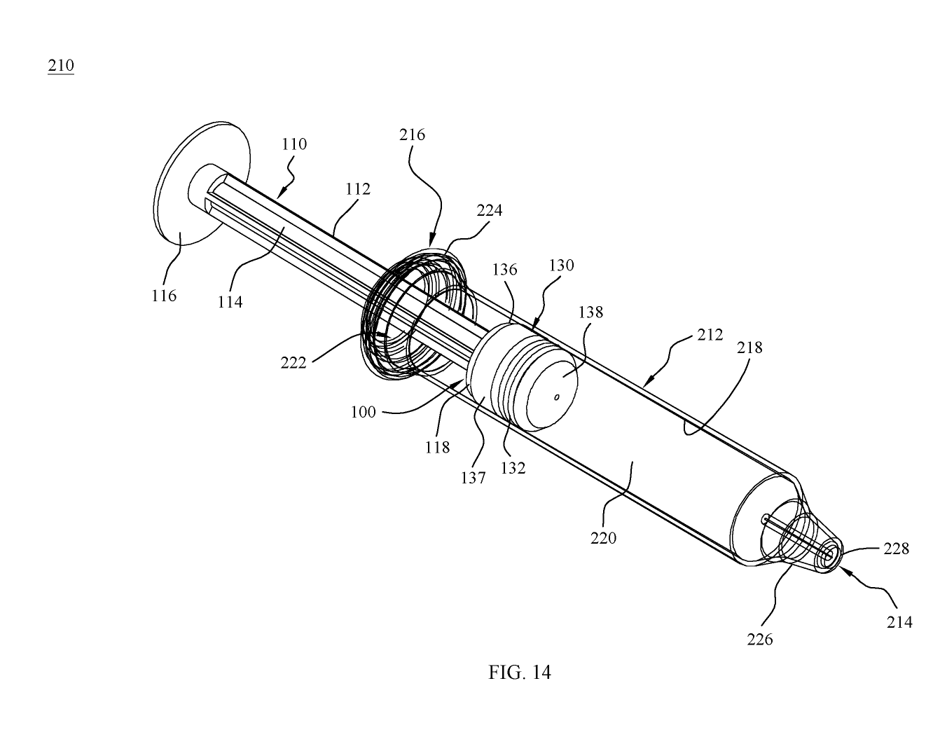

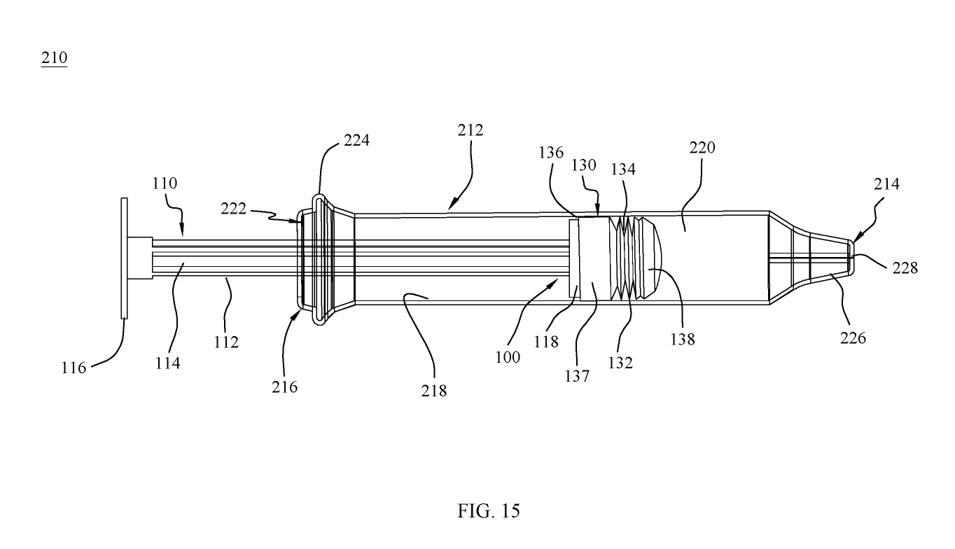

Once the force being applied to the piston 130 by the plunger rod 110 is sufficient to generate a resultant force great enough to overcome the inherent frictional wall forces on the corresponding piston 130, the piston 130 begins to move toward the first end 214 of the container 212. As the piston 130 moves, the fluid resistance in the injection mechanism (not shown) is overcome and the medication is forced out of the injection mechanism for delivery to the patient. FIGS. 13 and 14 illustrate the position of the piston 130 at the beginning of an injection to the patient and FIG. 15 illustrates the position of the piston 130 near the end of the injection to the patient. After an injection, the plunger rods 110, 140, 150, 160, 170, 180, 190 may be sterilized and used for another injection. Since the plunger rods 110, 140, 150, 160, 170, 180, 190 do not come into contact with the medication during injection they may be reusable.

As may be recognized by those of ordinary skill in the art based on the teachings herein, numerous changes and modifications may be made to the above-described and other embodiments of the present invention without departing from the scope of the invention. For example, injection systems may include more or fewer components or features than the embodiments as described and illustrated herein. For example, the plunger rods 110, 140, 150, 160, 170, 180, 190 may all be used interchangeably and in alternative combinations as would be modified or altered by one of skill in the art. Accordingly, this detailed description of the currently-preferred embodiments is to be taken as illustrative, as opposed to limiting of the invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

Having thus described the preferred embodiments, the invention is now claimed to be:

1. A plunger rod, comprising:
a shaft with a first end and a second end;
an injection member coupled to the shaft at the first end; and
a contact member coupled to the shaft at the second end.

2. The plunger rod of claim 1, further comprising:
a support ring positioned adjacent to the contact member on a distal side of the contact member.

3. The plunger rod of claim 1, wherein the contact member comprises:
at least one opening extending through the contact member.

4. The plunger rod of claim 1, wherein the shaft further comprises:
at least one groove along the length of the shaft extending from the contact member to a position near the first end.

5. A plunger rod system, comprising:
a piston; and
a plunger rod positioned adjacent to the piston.

6. The plunger rod system of claim 5, wherein the piston comprises:
a first base portion at a first end of the piston;
a second base portion at a second end of the piston;
at least one flange member positioned between the first base portion and the second base portion; and
a contact surface on an end of the first base portion.

7. The plunger rod system of claim 6, wherein the piston further comprises:
grooves positioned adjacent to each of the at least one flange members.

8. The plunger rod system of claim 6, wherein the plunger rod comprises:
a shaft with a first end and a second end;
an injection member coupled to the shaft at the first end; and
a contact member coupled to the shaft at the second end.

9. The plunger rod system of claim 8, wherein the contact member of the plunger rod is positioned adjacent to the contact surface of the first base portion of the piston.

10. The plunger rod system of claim 8, wherein the plunger rod further comprises:
a support ring positioned adjacent to the contact member on a distal side of the contact member.

11. The plunger rod system of claim 8, wherein the contact member comprises:
at least one opening extending through the contact member

12. The plunger rod system of claim 8, wherein the shaft further comprises:
at least one groove along the length of the shaft extending from the contact member to a position near the first end.

13. An injection system, comprising:
a container with a first end and a second end;
a piston positioned within the container; and
a plunger rod positioned within the container adjacent to the piston.

14. The injection system of claim 13, wherein the container further comprises:
an opening at the second end;
a chamber extending into the container from the opening toward the first end;
a sidewall formed by the chamber; and
an injection tip at the first end of the container.

15. The injection system of claim 14, wherein the container further comprises:
a flange positioned around an exterior surface of the container at the second end; and
an injection mechanism opening at the first end extending through the injection tip and into the chamber.

16. The injection system of claim 14, wherein the piston comprises:
a first base portion at a first end of the piston;
a second base portion at a second end of the piston;
at least one flange member positioned between the first base portion and the second base portion; and
a contact surface on an end of the first base portion;
wherein a side exterior surface of the first base portion, a side exterior surface of the second base portion, and the at least one flange engage the sidewall of the container.

17. The injection system of claim 16, wherein the plunger rod comprises:
a shaft with a first end and a second end;
an injection member coupled to the shaft at the first end; and
a contact member coupled to the shaft at the second end;
wherein a side exterior surface of the contact member engages the sidewall of the container.

18. The injection system of claim 17, wherein the contact member of the plunger rod is positioned adjacent to the contact surface of the first base portion of the piston in the chamber of the container.

19. The injection system of claim 18, wherein the plunger rod further comprises:
a support ring positioned adjacent to the contact member on a distal side and the support ring engages the sidewall of the container.

20. A method of assembling an injection system, comprising:
obtaining a container, a piston, and a plunger rod;
inserting the piston into an opening in a second end of the container; and
inserting the plunger rod into the opening in the second end of the container and positioning the plunger rod adjacent to the piston.
